(19) United States
(12) Patent Application Publication
(10) Pub. No.: US 2017/0072417 A1
(43) Pub. Date: Mar. 16, 2017

(54) METHOD AND DISPENSER DEVICE FOR DEPOSITING A SUBSTANCE ON A TARGET SUBSTRATE

(71) Applicant: SCIENION AG, Berlin (DE)

(72) Inventors: Holger EICKHOFF, Berlin (DE); Guido BARED, Berlin (DE)

(73) Assignee: SCIENION AG, Berlin (DE)

(21) Appl. No.: 15/120,182

(22) PCT Filed: Feb. 26, 2014
(86) PCT No.: PCT/EP2014/000506

§ 371 Date: Aug. 19, 2016

(51) Int. Cl. B05B 12/12; B05B 1/02; B05B 17/06; B05B 13/02; A61B 17/20; G06K 9/78; G06K 9/62; G06T 7/00; H04N 7/18; A61F 2/915; B01L 3/02; B05D 1/30

(52) U.S. Cl. CPC B05B 12/124; B01L 3/0268; B05B 1/02; B05B 17/0607; B05B 13/02; B05D 1/30; G06K 9/78; G06K 9/62; G06T 7/004; G06T 7/0012; H04N 7/181; A61F 2/915; A61B 17/205; B01L 2200/14; G06T 2207/30052

(57) ABSTRACT

A method of depositing at least one substance on a target substrate (1) comprises the step of operating at least one droplet dispenser (21) such that droplets (2) including the at least one substance are deposited on the target substrate (1), wherein the target substrate (1) has a substrate surface including spatially delimited receptacle sections (3) being arranged for accommodating the droplets (2), and the at least one droplet dispenser (21) is controlled in dependency on the locations of the receptacle sections (3) such that the droplets (2) are directed onto the receptacle sections (3). Furthermore, a dispenser device (100) for depositing at least one substance on a target substrate (1) is described.

### FIELD OF THE INVENTION

The present invention relates to a method of depositing at least one substance on a target substrate, in particular to a method of depositing droplets using at least one droplet dispenser. Furthermore, the invention relates to a dispenser device, which is adapted for depositing at least one substance on a target substrate, in particular including at least one droplet dispenser. Applications of the invention are available in the fields of loading substances to substrates, modifying substrate surfaces and/or functionalizing substrate surfaces, in particular in biology, biochemistry, chemistry or medicine.

### BACKGROUND ART

The deposition of liquid droplets on substrates using a droplet dispenser is generally known. For example, droplets are created from a reservoir and transferred through a nozzle to the substrate by the effect of a piezoelectric drive (piezoelectric droplet dispenser). If an array of droplets is to be deposited on the substrate, the piezoelectric drive is periodically activated, while the dispenser and the substrate are translated relative to each other so that a series of droplets is transferred to the substrate. The geometric positions of the droplets within a droplet array are determined by the dispensing frequency (droplet rate) of the dispenser and by the velocity of the mutual translation of the dispenser and the substrate. With a further example, liquid droplets can be loaded to the tip of a dispenser needle and transferred to the substrate by a contact of the needle tip with the substrate (contact spotter). With the contact spotter, multiple droplets can be deposited by serially operating one or more dispenser needles using a fixed or moving substrate.

The conventional droplet dispensing techniques typically are restricted to the deposition of droplets on plane substrate surfaces. For example, single droplets or droplet arrays are deposited on a plane glass slide or on plane cup bottoms in a microtitre plate. With the piezoelectric droplet dispenser, variations of the dispensing frequency and/or the droplet velocity may occur. Corresponding variations of the droplet positions can be tolerated with most of the applications if the droplets are deposited on the plane substrate.

It is practically known that modulating the droplet shape may enhance the deposition accuracy on the substrate. However, if the substrate has a structure with particular receptacle sections and the droplets have to be deposited even on the receptacle sections of the structured substrate, the conventional dispensing techniques may yield unacceptable results. Due to variations of the dispensing frequency and/or the velocity of mutual translation, droplets can be deposited on a portion of the receptacle sections only and even in spaces between the receptacle sections.

The application of a contact spotter is restricted to a plane substrate as well. The contact spotter requires a stable substrate surface. Delimited receptacle sections, e. g. protruding from the substrate surface, could be destroyed by the needle, or they could destroy the needle tip.

A particular example of a structured substrate to be loaded with a substance is a so-called needle patch, which comprises an array of needles on a solid carrier. Needle patches have been proposed as a tool for injecting substances for medical treatments, like e. g. vaccines or pain therapy, into the skin of a patient. The substance to be injected is applied to the needle tips. Conventionally, this application is obtained by immersing (dipping) the needles into a reservoir of the substance. However, the immersing technique does not allow an application of the substance exclusively to the tips of the needles, resulting in an essential disadvantage in terms of large substance consumption. This is a problem even with the application of expensive or highly toxic drugs to needle patches where it is crucial to prevent over dosage, but also with other tasks of depositing substances on structured substrates. In addition, the conventional dipping method does not allow loading of needle patches with multiple substances.

### Objective of the Invention

The objective of the invention is to provide an improved method of depositing at least one substance on a target substrate, wherein disadvantages of conventional techniques are avoided. In particular, the objective of the invention is to provide an improved method of depositing the at least one substance, wherein the method allows a precise and reproducible deposition at particular geometric positions on the target substrate. Furthermore, the objective of the invention is to provide an improved dispenser device for depositing at least one substance on a target substrate, which is capable of avoiding disadvantages of conventional techniques. In particular, the dispenser device is to be capable to deposit the at least one substance on delimited receptacle sections of a target substrate surface.

### SUMMARY OF THE INVENTION

These objectives are solved with a droplet dispensing method and a droplet dispenser device comprising the features of the independent claims, respectively. Advantageous embodiments and applications of the invention are defined in the dependent claims.

According to a first general aspect of the invention, a method of depositing at least one substance on a target substrate using at least one droplet dispenser is provided, wherein a substrate surface of the target substrate comprises a plurality of spatially delimited receptacle sections and the at least one droplet dispenser is controlled such that droplets including at least one substance are deposited on the receptacle sections.

According to a second general aspect of the invention, the above objective is solved by a droplet dispenser device comprising one or more dispenser heads with at least one droplet dispenser, a substrate support device being adapted for accommodating a target substrate and a control device being adapted for controlling an operation of the at least one droplet dispenser. According to the invention, the control device is adapted for controlling the at least one droplet dispenser such that the droplets dispensed by the at least one droplet dispenser are deposited at predetermined spatially delimited receptacle sections of the target substrate.

According to the invention, the at least one droplet dispenser is operated at predetermined dispenser positions, which are selected such that the droplets are placed on the receptacle sections, while a deposition of droplets in spacing between the receptacle sections preferably is avoided. The droplets are deposited on the target substrate with a predetermined geometric pattern, which is equal to the geometric arrangement of the receptacle sections. Contrary to conventional techniques, wherein the droplet arrays is created in dependency on a dispensing frequency of the dispenser and a mutual velocity of the dispenser and the target substrate, the invention teaches the targeted deposition of droplets on the target substrate, in particular on the receptacle sections, using the droplet dispenser. The inventors have found that the conventional control of the dispensing frequency and velocity does not allow a deposition on delimited receptacle sections with sufficient precision and reliability. With the inventive control of the dispenser in dependency on the positions of the receptacle sections, at least one substance included in the droplets can be deposited with precision and reproducibility. Thus, preferably, the droplet dispenser is controlled in dependency on the locations of the receptacle sections, in particular for depositing the droplets exclusively on the receptacle sections. With preferred examples, the locations of the receptacle sections can be provided by means of a visual detection system and/or by a stored map, as further outlined below.

Generally, the droplet dispenser is a dispensing component, which is capable of creating droplets and driving the droplets via a distance towards the target substrate, wherein the dispenser does not contact the target substrate. Preferably, the dispenser comprises a piezoelectric dispenser, including a liquid reservoir, a piezoelectric drive unit and a dispenser nozzle. The droplet dispenser has a characteristic droplet path of the droplet from a dispenser nozzle. The droplet path can be obtained e. g. with a calibration and/or using the image data collected during the dispensing operation.

Multiple applications of the inventive droplet deposition are available, which can be selected by a user in dependency on a particular dispensing task. In particular, one single dispenser head with one single dispenser, one single dispenser head with multiple dispensers, or multiple dispenser heads with one single dispenser or multiple dispensers can be used for depositing one single droplet or multiple droplets on each or selected ones of the receptacle sections, wherein the droplets include one single substance or different substances. According to a preferred embodiment of the invention, different substances can be deposited at different receptacle sections. According to a further preferred embodiment of the invention, the dispenser head includes multiple, e. g. two droplet dispensers supplying droplets along mutually crossing droplet paths. The droplet dispensers are arranged such that during dispensing operation the droplet paths hit one of the receptacle section. Droplets can be directed along the different droplet paths onto the receptacle section. With a preferred example, two droplets can be applied on two opposite sides of the receptacle section, e.g. a needle tip.

Advantageously, the inventors have found a plurality of operation parameters of the droplet dispenser, which can be controlled for directing the droplets even onto the receptacle sections. Preferably, the operation parameters are controlled during the dispensing operation of the droplet dispenser depositing a series of droplets on the target substrate. Accordingly, the dispenser is controlled by varying operation parameters during depositing a series or an array of droplets. Preferably, the following operation parameters are controlled. Each of the following operation parameters can be controlled alone, or two or more operation parameters can be controlled in combination.

According to a first variant, a droplet speed is adjusted by controlling the dispenser. To this end, the power of the droplet generating unit, in particular the drive voltage of the piezoelectric drive unit, is controlled. In particular, with optimizing the droplet speed, the deposition precision can be improved. For example, optimizing the droplet speed may include increasing or decreasing the velocity for improving the deposition precision. With a preferred example, the droplet speed can be controlled such that the droplets arrive with minimum kinetic energy at the receptacle sections. Advantageously, a distribution of the substance resulting from the impact of the droplet on the receptacle section (creation of splatters) is minimized. Preferably, the droplet speed is reduced below 5 m/s, particularly preferred below 3 m/s, e. g. to 1.5 m/s to 2.5 m/s.

Furthermore, the droplet frequency can be adjusted, e. g. for depositing subsequent droplets onto different receptacle sections or onto one single receptacle section. Advantageously, the droplet flight from the dispenser to the receptacle section can be stabilized by selecting the dispensing frequency. An increased frequency supports the stable creation of droplets with increased viscosity.

According to yet a further variant, a droplet shape can be controlled. The precision of the droplet deposition even on delimited receptacle sections can be improved if the geometric droplet shape is adapted to the shape of the receptacle section. The droplet shape can be influenced by the pulse shape, in particular width and drive voltage, of the piezoelectric drive unit. Preferably, the pulse shape can be selected such that the droplets have a longitudinal shape in z-direction, which results in an adaptation of the droplet shape to the shape of a longitudinal receptacle section.

A further variant of the invention comprises adjusting a droplet size, in particular a droplet diameter. Again, the precision of the droplet deposition can be improved by matching the droplet size to a characteristic dimension of the receptacle section, e. g. the diameter thereof. With a preferred example, the pulse width of the drive voltage of the piezoelectric drive unit influences the volume of the droplets. With increasing pulse width, increasing droplets can be dispensed.

According to a further modification, controlling the dispenser device may include adjusting a droplet viscosity. In particular, for depositing droplets on tip-shaped receptacle sections, increasing the droplet viscosity, compared e. g. with the viscosity of watery droplets, can increase the reproducibility of droplet deposition. Adherence of the deposited droplets on the receptacle sections can be increased with increasing droplet viscosity. Adjusting the droplet viscosity can be obtained e. g. by adding a solvent and/or a viscosity influencing substance to the liquid in the droplet dispenser.

Finally, as a further variant, a dispensing angle can be adjusted for controlling the dispenser. The dispensing angle is the angle of the movement path of the droplet relative to a normal direction of the target substrate surface. In particular, the dispensing angle can be adjusted in dependency on the orientation of the target substrate surface at the receptacle section relative to a vertical direction (z-axis). As an example, the substrate support device can be adapted for an inclination of the target substrate relative to the z-axis. Additionally or alternatively, the droplet dispenser can have a dispensing direction being inclined relative to the z-axis. Advantageously, the dispensing angle can be adjusted such that the droplets arrive with a grazing incidence at the receptacle sections. This allows a sloped deposition of the droplets on the receptacle sections. The adherence of the droplets, in particular on the needle-shaped receptacle sections, can be improved.

According to a preferred embodiment of the invention, the dispenser is controlled such that it is operated at particular dispensing coordinates, i. e. the positions at which the dispenser is operated are selected such that the droplets are deposited evenly at the receptacle sections. The droplet generating unit of the droplet dispenser is activated at the dispensing coordinates. Advantageously, various procedures for selecting the dispensing coordinates based on the positions of the receptacle sections are available. In addition to the positions of the receptacle sections, the droplet path (length, direction) of the droplet from the dispenser to the receptacle section taken into consideration for selecting the dispensing coordinates.

According to an advantageous embodiment of the invention, the dispensing coordinates are selected using a stored receptacle map. The receptacle map comprises data representing the geometric arrangement of the receptacle sections on the target substrate. The coordinates of the receptacle sections in a local coordinate system of the target substrate, e. g. the coordinates relative to target substrate boundaries, provide the receptacle map. The receptacle map can be stored in the dispensing device, in particular in the control device thereof. The dispensing coordinates are obtained by detecting a substrate position of the target substrate relative to the droplet dispenser and calculating the dispensing coordinates using the substrate position and the receptacle map. Preferably, the substrate position is detected using a first detector device, e. g. a first camera or another photosensor device, which is coupled with the droplet dispenser, e. g. fixed to the dispenser head, or with a dispenser head support. The embodiment using the receptacle map has particular advantages in terms of a simple data processing. The dispensing coordinates can be calculated with high speed on the basis of the stored receptacle map and the substrate position only.

According to an alternative embodiment, the dispensing coordinates are selected using detected receptacle positions of the receptacle sections relative to the droplet dispenser. This embodiment of the invention has particular advantages if droplets are deposited on target substrates having varying geometric positions of the receptacle sections on the target substrate and/or varying geometric orientations of the target substrate on a substrate support. Preferably, the receptacle positions are detected by collecting an image of the target substrate, recognizing the receptacle sections in the image and calculating the receptacle positions relative to the droplet dispenser. Thus, with a particularly preferred embodiment of the invention, the droplet dispenser is controlled in dependency on image data representing the receptacle positions. Again, according to a particularly preferred embodiment, the image is collected with a first detector device, e. g. a first camera or another photosensor device, which is coupled to the droplet dispenser, in particular attached to the dispenser head, or to the dispenser head support.

According to a further advantageous embodiment of the invention, a second detector device can be provided which is arranged for detecting flying droplets dispensed by the droplet dispenser. With this embodiment, the control device provides and/or corrects the dispensing coordinates based on one or more positions of dispensed droplets.

Advantageously, further process conditions are available for improving the deposition of the droplets on the receptacle sections. Preferably, deposition conditions of the target substrate, like a relative humidity in a substrate space adjacent to the target substrate surface and/or a temperature of the target substrate can be adjusted such that the droplets are dried immediately after being deposited on the receptacle sections. Accordingly, a distribution of the droplets around the receptacle sections is avoided as the drying results in an immobilization of the at least one substance included in the droplets.

With a preferred embodiment, the dispenser device may comprise a dispenser enclosure. By adjusting the relative humidity in the enclosure, the duration of drying the droplets on the receptacle sections can be influenced. With increasing drying speed, deposition of further droplets on the same receptacle section is facilitated. Additionally or alternatively, the target substrate can be subjected to a temperature adjustment. Again, the temperature of the target substrate can influence the duration of the drying.

With a preferred application of the invention, the target substrate is a solid plate having an exposed main surface including the receptacle sections. Preferably, the receptacle sections comprise structures projecting from the main surface and/or being recessed in the main surface of the target substrate. Preferably, each receptacle section has a characteristic dimension of an area exposed for accommodating the droplet, being below 1 mm, in particular below 100 μm, e. g. 50 μm or smaller. Furthermore, at least a portion of the surface of the receptacle sections can be provided with a functional coating, e. g. a hydrophobic coating. The hydrophobic coating can be obtained e. g. by silanization. Advantageously, the adherence of the substance on the receptacle sections can be optimized by the hydrophobic coating. Furthermore, the coverage of the receptacle section can be controlled by applying the functional coating to a specific part and not to the complete receptacle section.

As a particular advantage of the invention, droplets can be deposited on different types of receptacle sections. Preferred examples of projecting receptacle sections are needles, pyramids, cones, lamellas and/or parts thereof. With a particularly preferred application of the invention, the target substrate includes a needle array, e. g. for vaccination purposes. Advantageously, the invention allows a deposition of substances on the tips of projecting structures, wherein the tips have a characteristic dimension below 400 μm, in particular below 50 μm.

Recessed receptacle sections in the main surface comprise e. g. microfluidic channels. As an example, grooves in a solid body can be coated with substances during manufacturing a fluidic microsystem. According to yet another application, the receptacle sections may comprise filaments, which are arranged as three-dimensional network. With a particularly preferred embodiment of the invention, the target substrate comprises an implant device, like e. g. a stent, made of filaments. The invention provides a precise and reproducible deposition of substances even on the filaments.

According to a further advantageous embodiment of the invention, an image of the target substrate can be collected after depositing the droplets on the receptacle sections. Collecting the image can be used e. g. for testing the deposition result and/or for collecting position data for subsequent deposition steps. The imaging of the target substrate allows the implementation of a control loop, wherein operation parameters of the droplet dispenser as mentioned above can be controlled in dependency on image data such that the deposition of the droplets on the receptacle sections is obtained.

Preferably, the imaging of the target substrate includes an illumination with light having a wavelength selected in dependency on spectroscopic properties of the at least one substance deposited on the receptacle sections. As an example, if the substance has a strong absorption or reflection or fluorescence in certain spectral ranges, the illumination light can be selected in dependency on at least one of these spectroscopic properties, thus improving the test result obtained with the imaging step.

### DESCRIPTION OF THE DRAWINGS

Further advantages and details of the invention are described in the following with reference to the attached drawings, which show in

FIG. 1: a schematic sectional view of a preferred embodiment of a dispenser device according to the invention;

FIG. 2: a schematic perspective view of a needle array, e. g. for vaccination purposes;

FIG. 3: a flow chart illustrating further features of preferred embodiments of the dispensing method according to the invention;

FIG. 4: further examples of target substrates comprising delimited receptacle sections; and

FIG. 5: a schematic view of a further preferred embodiment of a dispenser device according to the invention.

### DESCRIPTION OF PREFERRED EMBODIMENTS

Features of preferred embodiments of the invention are described in the following with exemplary reference to a dispenser device having one single dispenser head with one single droplet dispenser, wherein the dispenser head is moveable relative to a target substrate. It is emphasized that the implementation of the invention is not restricted to this configuration, but correspondingly possible e. g. with a dispenser device having multiple dispenser heads and/or multiple droplet dispensers at the dispenser head. Furthermore, the substrate device can be moveable relative to the dispenser head. First and second detector devices with respective functions are described in an exemplary manner. Alternatively, only one detector device can be provided, e.g. fulfilling all described functions. While cameras are described below which have vertical fields of view, additionally or alternatively cameras can be used which have horizontal fields of view.

Furthermore, exemplary reference is made to a dispenser device having a piezoelectric dispenser. Details of the piezoelectric dispenser and the operation thereof are not described as far as they are known from conventional piezoelectric dispensers. Other types of contact-free droplet dispensers can be used as well, like e. g. a bubble-jet-dispenser.

Embodiments of the invention are described in the following with reference to an orthogonal coordinate system, including x- and y-axis extending in a target plane, and a z-axis perpendicular to the target plane. The direction of moving a droplet from the dispenser to the target substrate substantially is the negative z-direction (negative vertical direction).

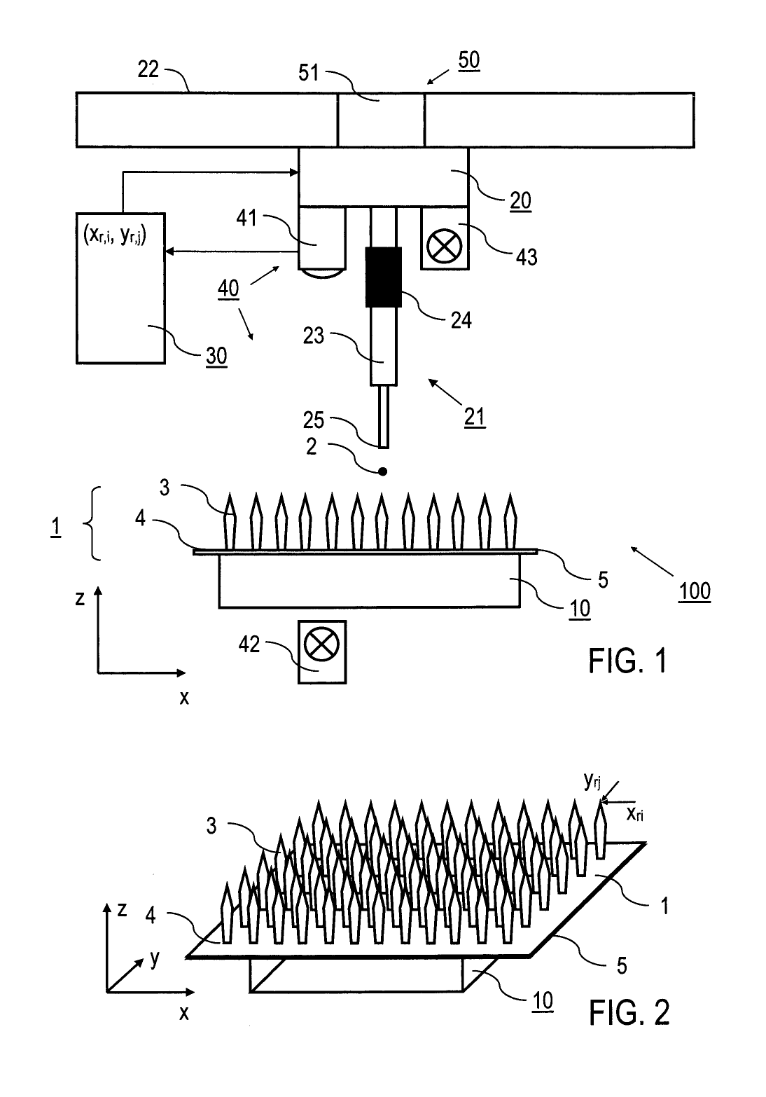

FIG. 1 schematically illustrates a dispenser device 100 comprising a substrate support device 10, a dispenser head 20 with a droplet dispenser 21, a control device 30, a first detector device 40 and a second detector device 50.

The substrate support device 10 is a substrate carrier platform, which is adapted for accommodating a target substrate 1. The target substrate 1 is secured to the substrate support device 10, e. g. with holding elements (not shown) and or by a vacuum. The substrate support device can be adapted for an inclination of the target substrate 1 relative to the z-axis, e. g. using piezoelectric drive elements (not shown).

The dispenser head 20 is carried by a dispenser head support 22. The substrate support device 10 with the target substrate 1 and the dispenser head 20 are moveable relative to each other, e. g. by translating the dispenser head 20 along the dispenser head support 22. The droplet dispenser 21 is a piezoelectric dispenser including a liquid reservoir 23, a piezoelectric drive unit 24 and a dispenser nozzle 25. In response to an activation of the piezoelectric drive unit 24, a droplet 2 is created and moved towards the substrate target 1. The droplet 2 has a volume of e. g. 300 pl, and the distance between the nozzle 25 and the target substrate 1 is e. g. 300 μm to 600 μm.

The substrate support device 10 and the dispenser head 20 with the dispenser 21 and the dispenser head support 22 can be structured as it is known from conventional dispenser devices, e. g. “sciFLEXARRAYER Sx Vacuum Holder”, manufacturer: Scienion AG, Germany.

The dispenser device 100 is adapted for depositing droplets 2 onto the target substrate 1 having multiple receptacle sections 3 connected with a base plate 4. The receptacle sections 3 comprise an array arrangement of needles as further illustrated in FIG. 2. The array arrangement comprises a matrix of straight columns and rows of receptacle sections 3. The target substrate 1 can be provided with additional marker elements (not shown). The marker elements can be adapted for facilitating the image recognition of the target substrate 1 with the first or second detector devices 40, 50.

With a preferred application of the invention, the target substrate 1 is a needle pad for vaccination or drug delivery purposes. With these examples, a few hundered up to several thousand needles are provided, which are made of e. g. Ti, Si or a ceramic. The needles have a longitudinal, polygonal shape as schematically illustrated. Depending on the structuring method for creating the receptacle sections 3, the needles may have a differing shape, e. g. a pyramid shape or a cone shape. Typically, the receptacle sections have a length in z-direction of about 300 μm, a cross-sectional dimension in the x-y plane of about 40 μm and a tip diameter of about 10 μm to 20 μm.

The tips of the receptacle sections 3 have receptacle coordinates x_{r,i},y_{r,j}, which represent the receptacle positions with a local coordinate system relative to the boundaries 5 of the target substrate 1 or in a global coordinate system relative to the dispenser head support 22. Depending on the structuring method used for manufacturing the target substrate 1, the positions of the receptacle sections 3 can deviate from ideal positions e. g. on a rectangular lattice. The real positions may result e. g. from bending receptacle sections 3 or from manufacturing tolerances. Furthermore, the substrate can be located on the substrate support device 10 such that the orientation of rows of receptacle sections 3 is rotated relative to the translation direction of the dispenser head 20. As a main advantage of the invention, the dispenser device 100 is capable of precisely depositing droplets 2 on the receptacle sections 3 by controlling the dispenser 21 even on these real positions.

The first detector device 40 is connected with the dispenser head 20. It comprises e. g. a first camera 41 (so-called head camera, CCD-camera). Preferably, the first camera 41 is arranged for detecting receptacle positions of the individual receptacle sections 3 relative to the dispenser 21, detecting a substrate position of the target substrate 1 relative to the dispenser 21, and/or collecting image data of the loaded target substrate 1 for testing or controlling purposes. The coordinates x_{r,i}, y_{r,j} can be obtained from an image collected with the first camera 41. In combination with the substrate position data, the first detector device 40 provides the receptacle positions relative to the position of the dispenser 21. Furthermore, with position data of the dispenser head 20 relative to the dispenser head support 22 and the image data obtained with the first camera 41, the substrate position of the target substrate 1 relative to the droplet dispenser 21 can be obtained.

The first detector device 40 is combined with one or two light sources 42, 43, which can be arranged above or below the target substrate 1. Preferably, two light sources 42, 43 having different illumination wavelengths are used. The illumination wavelengths are selected for optimizing the collection of an image of the receptacle sections 3 before and after the position of the droplets 2, respectively. As an example, the illumination wavelength of the light source 43 may be adapted for exciting fluorescence of a substance deposited on the receptacle sections 3.

The second detector device 50 comprises at least one second camera 51 (so-called droplet camera, CCD-camera), which is capable of detecting flying droplets 2 during dispensing operation of the droplet dispenser 21 and detecting one or more droplet positions (droplet path). The second camera 51 is connected e. g. with the dispenser head support 22. Alternatively, two droplet cameras can be provided for obtaining a three-dimensional image (3D image) of the droplet and detecting one or more droplet positions thereof. Detecting the droplet position(s) allows a further position control of the dispenser head 20. Optionally, the second detector device 50 can be used for detecting the droplet deposition in real time, and the control of the droplet dispenser 21 with the control device 30 can be corrected if necessary.

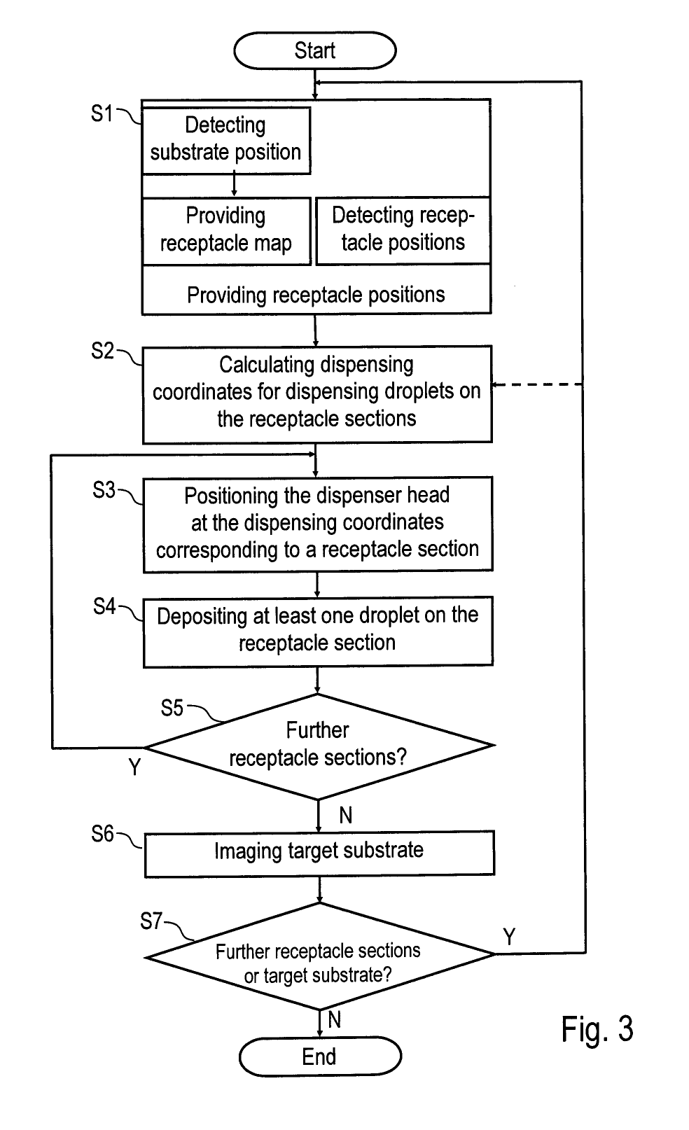

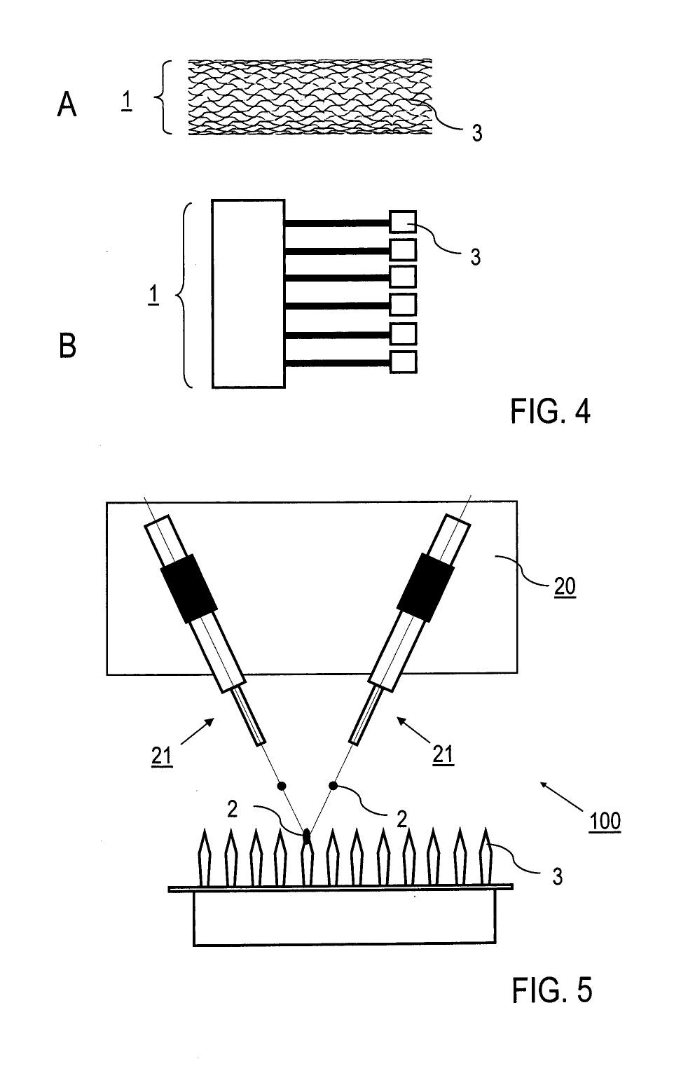

FIG. 3 illustrates features of a method of depositing droplets on a target substrate according to preferred embodiments of the invention. The method of FIG. 3 can be used e. g. for loading vaccination substances at the tips of a needle patch or depositing droplets on other types of target substrates, e. g. as shown in FIG. 4.

With step S1 of FIG. 3, receptacle positions of the receptacle sections 3 (see FIGS. 1, 2) are provided. In FIG. 3, two variants of providing the receptacle positions are illustrated, which can be used as alternatives or in combination. Firstly, the receptacle positions can be provided using a receptacle map, which is stored e. g. in the control device 30. The substrate position is detected e. g. with the first detector device 40. In combination with the receptacle map data, the substrate position provides the receptacle positions, e. g. in a global coordinate system. With the second variant, the receptacle positions are detected directly, using e. g. the first detector device 40.

With step S2, dispensing coordinates for dispensing droplets 2 onto the receptacle elements 3 are calculated, e. g. using the control unit 30. The calculated dispensing coordinates represent the positions, where the droplet dispenser 21 is to be operated for creating a droplet even on the receptacle sections 3, e. g. on the tips of the needles as shown in FIGS. 1 or 2. The dispensing coordinates are calculated in dependency on the receptacle positions and optionally in dependency on characteristics of the droplet path of the droplet from the nozzle 25 to the receptacle section 3. These droplet path characteristics are features of the droplet dispenser which can be obtained with a separate calibration measurement or using the image data collected with the second detector device 50.

With steps S3 an S4, the dispenser head 20 is positioned at the dispensing coordinates corresponding to one of the receptacle sections 3, and at least one droplet 2 is deposited on the receptacle element 3. The dispenser head 20 can be stopped or even moved during the dispensing step. Thus, a spot-on-the-fly method can be implemented for depositing single droplets without a stop-and-spot mode but by dispensing droplets while the head is moving. This embodiment of the invention leads to significant time savings.

Depending on the particular task of the dispensing method, only one receptacle section or multiple receptacle sections is/are coated with one or more droplets. Furthermore, the needles can be loaded with multiple substances, which is not possible with the conventional dipping method.

With step S5, the progress of depositing droplets on the target substrate is checked. If further droplets are to be deposited on further receptacle sections, steps S3 and S4 are repeated. Otherwise, the loaded target substrate is imaged with step S6 for testing purposes. In dependency on the result of the test, further droplets can be deposited on further receptacle sections, i. e. the steps S2 to S6 are repeated, possibly with modified operating parameters of the droplet dispenser 21, or another target substrate can be provided for droplet deposition. Otherwise, if the deposition task has been fulfilled, the process stops.

FIG. 4 illustrates further examples of target substrates 1. According to FIG. 4A, a side view of a 3D filament arrangement, e. g. of a stent, is shown. With the method of the invention, substances are to be deposited even on receptacle sections 3 comprising the filaments or crossings thereof, but not through the spacing between the filaments. The substances comprise e. g. antibiotic substances or coagulation suppressor substances. As described with reference to FIG. 3, the positions of the filaments can be provided using the position of the stent relative to a dispenser and the positions of the filaments, and the dispenser can be controlled such that droplets of a liquid are placed exclusively on the filaments.

FIG. 4B schematically illustrates another example, wherein the target substrate 1 comprises a micro-mechanical component, wherein receptacle sections 3 are located at the end of micro-mechanical levers. Again, the dispenser is controlled such that droplets are deposited on the receptacle sections 3 only.

FIG. 5 schematically illustrates features of a further embodiment of a dispenser device 100 according to the invention, wherein a dispenser head 20 carrying two dispensers 21 is used. The droplet paths of the dispensers 21 intersect each other. The droplet dispensers 21 are synchronously operated under control of the control device 30 as described above when both droplet paths hit the receptacle section 3 (target needle). With this embodiment, two droplets 2 are commonly driven towards the receptacle section 3 thus covering the receptacle section 3 from two sides.

The features of the invention disclosed in the above description, the drawings and the claims can be of significance both individually as well as in combination for the realization of the invention in its various embodiments.

1. A method of depositing at least one substance on a target substrate, comprising the step of:
operating at least one droplet dispenser such that droplets including the at least one substance are deposited on the target substrate, wherein
the target substrate has a substrate surface including spatially delimited receptacle sections being arranged for accommodating the droplets, and
the at least one droplet dispenser is controlled in dependency on locations of the receptacle sections such that the droplets are directed onto the receptacle sections.

2. The method according to claim 1, wherein
the at least one droplet dispenser is controlled by adjusting at least one of a droplet speed, a droplet frequency, a droplet shape, a droplet diameter, a droplet viscosity, and a dispensing angle.

3. A method according to claim 1, wherein
the at least one droplet dispenser is controlled using dispensing coordinates which are selected such that the droplets are deposited on the receptacle sections.

4. The method according to claim 3, wherein selecting the dispensing coordinates includes the steps of
storing a receptacle map representing a geometric arrangement of the receptacle sections on the target substrate,
detecting a substrate position of the target substrate relative to the at least one droplet dispenser using a first detector device, and
providing the dispensing coordinates based on the substrate position and the stored receptacle map.

5. The method according to claim 3, wherein selecting the dispensing coordinates includes the steps of
detecting receptacle positions of the receptacle sections relative to the at least one droplet dispenser using a first detector device, and
providing the dispensing coordinates based on the detected receptacle positions.

6. The method according to claim 5, wherein the step of detecting the receptacle positions includes the steps of
collecting an image of the target substrate,
recognizing the receptacle sections in the image, and
calculating the receptacle positions relative to the at least one droplet dispenser.

7. The method according to claim 5, wherein
the first detector device includes a first camera.

8. The method according to claim 1, including the step of
detecting droplets dispensed by the at least one droplet dispenser with a second detector device at least one second camera.

9. The method according to claim 1, including the step of
setting deposition conditions of the target substrate, comprising at least one of relative humidity in a substrate space including the substrate surface and temperature of the target substrate, such that the droplets, are dried immediately after being deposited on the receptacle sections.

10. The method according to claim 1, including at least one of the features
the step of operating the at least one droplet dispenser is repeated for depositing at least one of different substances and multiple quantities of one substance at the receptacle sections, and
the step of operating the at least one droplet dispenser includes depositing different substances at different of the receptacle sections.

11. The method according to claim 1, wherein
multiple droplet dispensers are synchronously operated such that multiple droplets are directed along different droplet paths onto at least one of the receptacle sections.

12. The method according to claim 1, wherein the receptacle sections include at least one of
structures projecting from a main surface of the target substrate,
at least one of needles, pyramids, cones, lamellas and parts thereof, projection from a main surface of the target substrate,
structures recessed in a main surface of the target substrate,
microfluidic channels recessed in a main surface of the target substrate,
filaments arranged as a two- or three-dimensional network,
micromechanical components,
cantilevers, and
hydrophobic coatings provided on the receptacle sections.

13. The method according to claim 12, wherein the target substrate includes
a needle array for vaccination purposes,
an implant device,
a stent, or
a micro-mechanical component.

14. The method according to claim 1, wherein
the receptacle sections have a characteristic lateral dimension below 100 μm.

15. The method according to claim 1, including the further step of
imaging the target substrate after depositing the droplets on the receptacle sections.

16. The method according to claim 15, wherein
the target substrate is illuminated with illumination light having a wavelength selected in dependency on spectroscopic properties of the at least one substance deposited on the target substrate.

17. A dispenser device, being arranged for depositing at least one substance on a target substrate, comprising:
a substrate support device being arranged for accommodating the target substrate,
a dispenser head including at least one droplet dispenser, wherein the substrate support device and the dispenser head can be positioned relative to each other such that the at least one droplet dispenser is arranged above the target substrate, and
a control device being arranged for operating the at least one droplet dispenser such that droplets including the at least one substance are deposited on the target substrate, wherein
the control device is configured for controlling the at least one droplet dispenser in dependency on the locations of the receptacle sections such that the droplets are deposited at spatially delimited receptacle sections of the target substrate.

18. The dispenser device according to claim 17, wherein
the control device is configured for adjusting at least one of a droplet speed, a droplet frequency, a droplet shape, a droplet diameter, a droplet viscosity, and a dispensing angle.

19. The dispenser device according to claim 17, wherein
the control device is configured for selecting dispensing coordinates and operating the at least one droplet dispenser at the selected dispensing coordinates such that the droplets are deposited at the receptacle sections.

20. The dispenser device according to claim 19, wherein
the control device includes a storage portion being arranged for storing a receptacle map representing a geometric arrangement of the receptacle sections on the target substrate,
a first detector device is arranged for detecting a substrate position of the target substrate relative to the at least one droplet dispenser, and
the control device is arranged for providing the dispensing coordinates based on the substrate position and the stored receptacle map.

21. The dispenser device according to claim 19, wherein
a first detector device is arranged for detecting receptacle positions of the receptacle sections relative to the at least one droplet dispenser, and
the control device is arranged for providing the dispensing coordinates based on the substrate position and the detected receptacle positions.

22. The dispenser device according to claim 21, wherein
the control device is arranged for an image recognition and for calculating the receptacle positions relative to the droplet dispenser.

23. The dispenser device according to claim 20, wherein
the first detector device includes a first camera being coupled with the at least one droplet dispenser or a dispenser head support.

24. The dispenser device according to claims 17, further comprising
a second detector device being arranged for detecting droplets dispensed by the at least one droplet dispenser, and
the control device is arranged for providing dispensing coordinates based on one or more positions of dispensed droplets.

25. The dispenser device according to claim 22, wherein
the second detector device includes at least one second camera coupled with the droplet dispenser or a dispenser head support.

26. The dispenser device according to claim 17, wherein
the dispenser head includes multiple droplet dispensers being arranged for directing droplets along different droplet paths onto at least one of the receptacle sections.

27. The method according to claim 1, wherein
the receptacle sections have a characteristic lateral dimension below 50 μm.
